(19) United States
(12) Patent Application Publication
(10) Pub. No.: US 2016/0235765 A1
(43) Pub. Date: Aug. 18, 2016

(54) Methods of Managing Eye Pain and Compositions Related Thereto

(71) Applicant: EMORY UNIVERSITY, Atlanta, GA (US)

(72) Inventors: Timothy W. Olsen, Atlanta, GA (US); Sonia Mehta, Wilmington, DE (US)

(73) Assignee: Emory University, Atlanta, GA (US)

(21) Appl. No.: 15/024,966

(22) PCT Filed: Sep. 25, 2014
(86) PCT No.: PCT/US2014/057450

§ 371 Date: Mar. 25, 2016

Related U.S. Application Data
(60) Provisional application No. 61/883,410, filed on Sep. 27, 2013.

(51) Int. Cl. A61K 31/573; A61K 45/06; A61K 31/546; A61K 9/00; A61K 31/445

(52) U.S. Cl. CPC A61K 31/573; A61K 9/0048; A61K 31/445; A61K 31/546; A61K 45/06

(57) ABSTRACT

This disclosure relates to methods of managing eye pain, for example, due to eye surgery. In certain embodiments, the disclosure relates to methods comprising administering a local anesthetic in combination with a steroid to the space around the eye in a subject in need thereof.

### CROSS-REFERENCE TO RELATED APPLICATIONS

This application claims priority to U.S. Provisional Application No. 61/883,410 filed the 27 Sep. 2013, hereby incorporated by reference in its entirety.

### GOVERNMENT ACKNOWLEDGMENT

This invention was made with government support under a grant from Research to Prevent Blindness number P30 EY006360 awarded by the National Institutes of Health. The Government has certain rights in the invention.

### BACKGROUND

Eye pain due to injury or surgery can be long lasting. A local anesthetic administered at the beginning of eye surgery is effective in preventing a patient from feeling pain during the operation. However, significant pain after the anesthetic has worn off is common For example, some patients experience mild to severe pain after LASIK surgery for several days. In more invasive eye procedures such as vitreoretinal surgery, a longer lasting anesthetic, such as bupivacaine is administered. However, severe eye pain over the next couple of days is typical. Narcotics may be prescribed for relief However, aside from the risk of addiction, narcotics cause undesirable post-operative nausea. Thus, there is a need for improved therapies that allows for longer periods of eye pain relief.

Fekrat et al. showed that 56% of patients undergoing vitreoretinal surgery had significant post-operative pain in the first 5 hours following surgery. Retina 2001; 21(6):627-632.

Er showed analyzed intraoperative ketorolac and eye pain after vitreoretinal surgery in a prospective, randomized, placebo-controlled study. Retina 2004 Feb; 24(1):182-3.

Mandelcorn et al. analyzed risk factors for pain and nausea following retinal and vitreous surgery under conscious sedation. Can J Ophthalmol 1999 Aug; 34(5): 281-5.

Covino and Wildsmith analyzed the clinical pharmacology of local anesthetics in neural blockage. Covino B G, Wildsmith J A W (1998). Clinical Pharmacology of Local Anesthetics in Neural Blockage in Clinical Anesthesia and Management of Pain, M J Cousins and P O Iridenbaugh, eds (pp 97-128). Philadelphia: Lippincott.

Song et al. report comparison of the effects of intravitreal triamcinolone acetonide and bevacizumab injection for diabetic macular edema. Korean J Ophthalmol, 2011, 25(3):156-60.

References cited herein are not an admission of prior art.

### SUMMARY

This disclosure relates to methods of managing eye pain, for example, due to eye surgery. In certain embodiments, the disclosure relates to methods comprising administering a local anesthetic in combination with a corticosteroid to the space around the eye in a subject in need thereof.

In certain embodiments, the disclosure relates to methods reducing, treating, or preventing eye pain comprising administering a local anesthetic in combination with a corticosteroid to the space around the eye in a subject in need thereof wherein the administration is performed before, during or after eye surgery optionally in combination with administering an antibiotic as the third component.

In certain embodiments, the space around the eye is selected from periorbital space, retrobulbar block, peribulbar block, subtenon's capsule area, orbicularis oculi muscle, superior, inferior, medial, lateral recuts muscle, superior, inferior oblique muscle, levator palpebrae superioris muscle, and in front of the tragus of the ear.

In certain embodiments, the local anesthetic is selected from bupivacaine, lidocaine, xylocaine, proxymetacaine, paracaine, and tetracaine, combinations, salts or prodrugs thereof.

In certain embodiments, the steroid is selected from triamcinolone, triamcinolone diacetate, triamcinolone acetonide, triamcinolone hexacetonide, cortisone, hydrocortisone, prednisone, prednisolone, methylprednisolone, fludrocortisone, beclomethasone, dexamethasone, and betamethasone, combinations, salts, or prodrugs thereof.

In certain embodiments, the antibiotic is selected from a cephalosporin, cefuroxime, cephtriaxone moxifloxacin, vancomycin, gentamycin, levofloxacin, polymyxin B, sulfamethoxazole, gatifloxacin, chloramphenicol, sulphadimidine, and penicillin, combinations, salts, or prodrugs thereof.

In certain embodiments, any of the methods disclosed herein may be accompanied by administering adrenaline and/or hyaluronidase in combination with the anesthetic.

In certain embodiments, the subject is diagnosed with retinal detachment, macular hole, epiretinal membrane, cataract, uveitis, intra- or peri-ocular tumor, proliferative vitreoretinopathy, retinoschisis vitreous hemorrhage, diabetic retinopathy, subretinal hemorrhage, or neovascular glaucoma.

In certain embodiments, the disclosure relates to methods of reducing or preventing postoperative eye pain comprising administering and effective amount of bupivacaine, salt or prodrug thereof in combination with triamcinolone, salt or prodrug thereof, and an antibiotic into an area located behind the globe of the eye in a subject in need thereof.

In certain embodiments, 3 to 9 mg of bupivacaine or 6 to 9 mg of bupivacaine is administered, 90 to 110 mg of cefazolin is administered, and 20 to 60 mg of triamcinolone or 50 to 60 mg of triamcinolone is administered.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

In certain embodiments, a pharmaceutical agent, which may be in the form of a salt or prodrug, is administered in methods disclosed herein that is specified by a weight. This refers to the weight of the recited compound. If in the form of a salt or prodrug, then the weight is the molar equivalent of the corresponding salt or prodrug.

It must be noted that, as used in the specification and the appended claims, the singular forms “a,” “an,” and “the” include plural referents unless the context clearly dictates otherwise.

As used herein, the term “combination with” when used to describe administration with an additional treatment means that the agent may be administered prior to, together with, or after the additional treatment, or a combination thereof.

“Subject” refers any animal, preferably a human patient, livestock, or domestic pet.

As used herein, the terms “prevent” and “preventing” include the prevention of the recurrence, spread or onset. It is not intended that the present disclosure be limited to complete prevention. In some embodiments, the onset is delayed, or the severity is reduced.

As used herein, “salts” refer to derivatives of the disclosed compounds where the parent compound is modified making acid or base salts thereof Examples of salts include, but are not limited to, mineral or organic acid salts of basic residues such as amines, alkylamines, or dialkylamines; alkali or organic salts of acidic residues such as carboxylic acids; and the like. In preferred embodiment the salts are conventional nontoxic pharmaceutically acceptable salts including the quaternary ammonium salts of the parent compound formed, and non-toxic inorganic or organic acids. Preferred salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. One example of a prodrug is compound in which a carboxylic acid group is converted to an ester such as a methyl or ethyl ester.

Methods of Use

This disclosure relates to methods of managing eye pain, for example, due to eye surgery. In certain embodiments, the disclosure relates to methods comprising administering a local anesthetic in combination with a steroid to the space around the eye in a subject in need thereof

In certain embodiments, the disclosure relates to methods reducing, treating, or preventing eye pain comprising administering a local anesthetic in combination with a steroid to the space around the eye in a subject in need thereof wherein the administration is performed before, during or after eye surgery optionally in combination with administering an antibiotic as the third component.

In certain embodiments, the space around the eye is selected from periorbital space, retrobulbar block, peribulbar block, orbicularis oculi muscle, superior, inferior, medial, and lateral rectus muscle, superior, inferior oblique muscle, levator palpebrae superioris muscle, and in front of the tragus of the ear.

The term “periorbital space” refers to the tissues and muscles surrounding or lining the eyeball. The “retrobulbar block” refers to the extraocular muscles behind the eyeball containing ciliary nerves, the ciliary ganglion, and cranial nerves. Injection of local anesthetic into the retrobulbar block results in akinesia of the extraocular muscles preventing movement of the globe. The “Peribulbar block” refers to muscle above and below the orbit containing orbicularis oculi muscle containing ciliary and cranial nerves.

In certain embodiments, the eye surgery is vitreoretinal surgery, vitrectomy, scleral buckle, anterior vitrectomy, pars plana vitrectomy (PPV), pan retinal photocoagulation, retinal detachment repair, pneumatic retinopexy, retinal cryopexy, cyclocryotherapy, macular hole repair, partial lamellar sclerouvectomy, partial lamellar sclerocyclochoroidectomy, partial lamellar sclerochoroidectomy, posterior sclerotomy, radial optic neurotomy, macular translocation surgery, cataract surgery, cataract extraction, glaucoma surgery, canaloplasty, refractive surgery, keratomilleusis, keratoplasty, automated lamellar keratoplasty, laser photocoagulation, laser eye surgery, laser assisted in-situ keratomileusis (LASIK), laser assisted sub-epithelial keratomileusis (LASEK), photorefractive keratectomy (PRK), laser thermal keratoplasty (LTK), conductive keratoplasty (CK), limbal relaxing incisions (LRI), astigmatic keratotomy (AK), radial keratotomy (RK), hexagonal keratotomy (HK), cornea transplant, epikeratophakia, intracorneal ring or corneal ring segments implant, contact lens implant, scleral expansion band (SEB) implant, anterior ciliary sclerotomy (ACS), scleral reinforcement surgery, cornea transplant, penetrating keratoplasty (PK), keratoprosthesis (KPro), phototherapeutic keratectomy (PTK), pterygium excision, corneal tattooing, iridectomy, strabismus, incision and drainage of styes and chalazions, oculoplastic procedures on the lids and eye removal.

The retina refers to the tissue lining the inner surface of the back of the eye that captures images that pass through the cornea and lens. Injury or trauma can tear the retina. A tear allows vitreous fluid to migrate under the retina, and retinal detachment can occur. Tumor growth on the tissue beneath the retina, choroidal melanoma, also causes retinal detachment. Retinal detachment and other retinal defects can be detected using fundus photography or ophthalmoscopy. Retinal tears are typically repaired with laser therapy or cryotherapy.

A posterior vitreous detachment (PVD) refers to a situation where the retina separates from the vitreous membrane. PVD often precedes a retinal detachment. Some symptoms of a PVD include: photopsia or flashes of light in the peripheral (outside of center) part of vision, an increase in the number of deposits and cells in vitrious humour, and a feeling of heaviness in the eye.

Proliferative vitreoretinopathy (PVR) refers to the formation of fibrous membrane on or around the retina, which may occur after retinal surgery. PVR can leads to inoperable retinal detachment.

Retinoschisis refers to the separation of retinal tissue typically causing cysts in the retina. It may be caused by a genetic defect [X-linked Juvenile Retinoschisis (XLRS)] or due to a natural degenerative process associated with aging.

Retinal tears can be repaired with laser therapy or cryotherapy. In scleral buckle surgery, or more bands are inserted, e.g. sewn to the sclera, configured such that the retina re-attaches. Retinal breaks are typically repaired by laser or cryotherapy prior to inserting the buckle. Typically, subretinal fluid is drained. In pneumatic retinopexy, a gas is injected into the eye after retinal breaks are repaired. The head of the subject is configured so that the bubble created by the gas rests against the retinal hole. The gas bubble must be kept in contact with the retinal hole for several days. In a vitrectomy, one removes vitreous gel from the eye, and the eye is optionally filled, e.g., with a gas or silicone oil, while retinal repairs heal after laser or cryotherapy.

Formulations

In certain embodiments, the disclosure relates to methods of reducing or preventing postoperative eye pain comprising administering and effective amount of a local anesthetic, such as bupivacaine, salt or prodrug, thereof in combination with a steroid such as triamcinolone, salt or prodrug thereof, and an antibiotic, such as cefazolin into an area located behind the globe of the eye in a subject in need thereof In certain embodiments, the disclosure relates to pharmaceutical composition comprising these compounds.

In certain embodiments, the composition comprises a mixture of bupivacaine 3 to 10 mg, or 5 to 20 mg, or 6 to 9 mg, or about 7.5 mg, cefazolin of 90 to 110 mg, or 50 to 150 mg or 50 to 200 mg, or about 100 mg, and triamcinolone 20 to 50 mg, or 20 to 60 mg, or 20 to 100 mg, or about 40 mg.

In certain embodiments, the composition comprises a mixture of bupivacaine, cefazolin, and triamcinolone which is in a ratio by weight of about 1:10:4 respectively or 0.5 to 1:8 to 12:3 to 6 respectively, or 0.5 to 1:6 to 14:1 to 8 respectively.

In certain embodiments, a subject undergoing scleral buckling surgery is given a mixture of bupivacaine 7.5 mg, cefazolin 100 mg, and triamcinolone 40 mg post-surgery to reduce pain.

In certain embodiments, a subject undergoing vitrectomy is given a mixture of bupivacaine 7.5 mg, cefazolin 100 mg, and triamcinolone 40 mg post-surgery to reduce pain.

In certain embodiments, a subject undergoing combined scleral buckle-vitrectomy surgery is given a mixture of bupivacaine 7.5 mg, cefazolin 100 mg, and triamcinolone 40 mg post-surgery to reduce pain.

In certain embodiments, a subject undergoing combined scleral buckle-vitrectomy surgery is given a 3-mL mixture of 1 mL 0.75% bupivacaine (7.5 mg/1 mL), 1 mL cefazolin (100 mg/mL), and 1 mL triamcinolone (40 mg/mL) post-surgery to reduce pain and the amount of block entering the retrobulbar space is estimated by estimating the volume of reflux.

In certain embodiments, the agent administered in combination with cefazolin and bupivacaine can be, but is not limited to, triamcinolone, triamcinolone benetonide, triamcinolone furetonide, triamcinolone hexacetonide and triamcinolone diacetate.

Pharmaceutical compositions disclosed herein can be in the form of pharmaceutically acceptable salts, as generally described below. Some preferred, but non-limiting examples of suitable pharmaceutically acceptable organic and/or inorganic acids are hydrochloric acid, hydrobromic acid, sulfuric acid, nitric acid, acetic acid and citric acid, as well as other pharmaceutically acceptable acids known per se (for which reference is made to the references referred to below).

When the compounds of the disclosure contain an acidic group as well as a basic group, the compounds of the disclosure can also form internal salts, and such compounds are within the scope of the disclosure. When a compound contains a hydrogen-donating heteroatom (e.g. NH), salts are contemplated to cover isomers formed by transfer of the hydrogen atom to a basic group or atom within the molecule.

Pharmaceutically acceptable salts of the compounds include the acid addition and base salts thereof Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases can also be formed, for example, hemisulphate and hemicalcium salts. For a review on suitable salts, see Handbook of Pharmaceutical Salts: Properties, Selection, and Use by Stahl and Wermuth (Wiley-VCH, 2002), incorporated herein by reference.

The compounds described herein can be administered in the form of prodrugs. A prodrug can include a covalently bonded carrier which releases the active parent drug when administered to a mammalian subject. Prodrugs can be prepared by modifying functional groups present in the compounds in such a way that the modifications are cleaved, either in routine manipulation or in vivo, to the parent compounds. Prodrugs include, for example, compounds wherein a hydroxyl group is bonded to any group that, when administered to a mammalian subject, cleaves to form a free hydroxyl group. Examples of prodrugs include, but are not limited to, acetate, formate and benzoate derivatives of alcohol functional groups in the compounds. Examples of structuring a compound as prodrugs can be found in the book of Testa and Caner, Hydrolysis in Drug and Prodrug Metabolism, Wiley (2006) hereby incorporated by reference. Typical prodrugs form the active metabolite by transformation of the prodrug by hydrolytic enzymes, the hydrolysis of amides, lactams, peptides, carboxylic acid esters, epoxides or the cleavage of esters of inorganic acids.

Pharmaceutical compositions typically comprise an effective amount of a compound and a suitable pharmaceutical acceptable carrier. The preparations can be prepared in a manner known per se, which usually involves mixing the at least one compound according to the disclosure with the one or more pharmaceutically acceptable carriers, and, if desired, in combination with other pharmaceutical active compounds, when necessary under aseptic conditions.

Generally, for pharmaceutical use, the compounds can be formulated as a pharmaceutical preparation comprising at least one compound and at least one pharmaceutically acceptable carrier, diluent or excipient and/or adjuvant, and optionally one or more further pharmaceutically active compounds.

Formulations containing one or more of the compounds described herein can be prepared using a pharmaceutically acceptable carrier composed of materials that are considered safe and effective and can be administered to an individual without causing undesirable biological side effects or unwanted interactions. The carrier is all components present in the pharmaceutical formulation other than the active ingredient or ingredients. As generally used herein “carrier” includes, but is not limited to, diluents, binders, lubricants, disintegrators, fillers, pH modifying agents, preservatives, antioxidants, solubility enhancers, and coating compositions.

Carrier also includes all components of the coating composition which can include plasticizers, pigments, colorants, stabilizing agents, and glidants. Delayed release, extended release, and/or pulsatile release dosage formulations can be prepared as described in standard references such as “Pharmaceutical dosage form tablets,” eds. Liberman et. al. (New York, Marcel Dekker, Inc., 1989), “Remington—The science and practice of pharmacy,” 20th ed., Lippincott Williams & Wilkins, Baltimore, Md., 2000, and “Pharmaceutical dosage forms and drug delivery systems,” 6th Edition, Ansel et al., (Media, Pa.: Williams and Wilkins, 1995). These references provide information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material can contain conventional carriers such as plasticizers, pigments, colorants, glidants, stabilization agents, pore formers and surfactants. Optional pharmaceutically acceptable excipients present in the drug-containing tablets, beads, granules or particles include, but are not limited to, diluents, binders, lubricants, disintegrants, colorants, stabilizers, and surfactants.

Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants can be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-beta-alanine, sodium N-lauryl-beta-iminodipropionate, myristoamphoacetate, lauryl betaine and lauryl sulfobetaine.

If desired, the tablets, beads, granules, or particles can also contain minor amount of nontoxic auxiliary substances such as wetting or emulsifying agents, dyes, pH buffering agents, or preservatives.

The compositions described herein can be formulation for modified or controlled release. Examples of controlled release dosage forms include extended release dosage forms, delayed release dosage forms, pulsatile release dosage forms, and combinations thereof.

The extended release formulations are generally prepared as diffusion or osmotic systems, for example, as described in “Remington—The science and practice of pharmacy” (20th ed., Lippincott Williams & Wilkins, Baltimore, Md., 2000). A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and Carbopol® 934, polyethylene oxides and mixtures thereof Fatty compounds include, but are not limited to, various waxes such as carnauba wax and glyceryl tristearate and wax-type substances including hydrogenated castor oil or hydrogenated vegetable oil, or mixtures thereof.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form comprising single or multiple units. Examples of multiple units include, but are not limited to, multilayer tablets and capsules containing tablets, beads, or granules. An immediate release portion can be added to the extended release system by means of either applying an immediate release layer on top of the extended release core using a coating or compression process or in a multiple unit system such as a capsule containing extended and immediate release beads.

Extended release tablets containing hydrophilic polymers are prepared by techniques commonly known in the art such as direct compression, wet granulation, or dry granulation. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In the congealing method, the drug is mixed with a wax material and either spray-congealed or congealed and screened and processed.

Delayed release formulations are created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition can be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and can be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including Eudragit® L30D-55 and L100-55 (soluble at pH 5.5 and above), Eudragit® L-100 (soluble at pH 6.0 and above), Eudragit® S (soluble at pH 7.0 and above, as a result of a higher degree of esterification), and Eudragits® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials can also be used. Multi-layer coatings using different polymers can also be applied.

The preferred coating weights for particular coating materials can be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition can include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates can also be used. Pigments such as titanium dioxide can also be used. Small quantities of an anti-foaming agent, such as a silicone (e.g., simethicone), can also be added to the coating composition.

Alternatively, each dosage unit in the capsule can comprise a plurality of drug-containing beads, granules or particles. As is known in the art, drug-containing “beads” refer to beads made with drug and one or more excipients or polymers. Drug-containing beads can be produced by applying drug to an inert support, e.g., inert sugar beads coated with drug or by creating a “core” comprising both drug and one or more excipients. As is also known, drug-containing “granules” and “particles” comprise drug particles that can or cannot include one or more additional excipients or polymers. In contrast to drug-containing beads, granules and particles do not contain an inert support. Granules generally comprise drug particles and require further processing. Generally, particles are smaller than granules, and are not further processed. Although beads, granules and particles can be formulated to provide immediate release, beads and granules are generally employed to provide delayed release.

### EXAMPLES

The Effects of Triamcinolone Acetonide on Retrobulbar Anesthesia

The effects of triamcinolone were evaluated in a study that was conducted in accordance with the IRB-approved protocol. Patients were randomized to two groups: Group A received a 3-mL mixture of 1 mL 0.75% bupivacaine (7.5 mg/1 mL), 1 mL cefazolin (100 mg/mL), and 1 mL triamcinolone (40 mg/mL). Group B received a 3-mL mixture of 1 mL 0.75% bupivacaine (7.5 mg/mL), 1 mL cefazolin (100 mg/mL), and 1 mL of balanced salt solution. A blunt cannula was used to deliver the injections into the retrobulbar space at the conclusion of the surgery after the conjunctiva was closed. The primary outcome was the pain score on postoperative day 1. Patients in group B required less pain medication than those in group A during the initial 24 hours following surgery (p=0.04). Full ocular motility was seen in 100% of the group A eyes on postoperative day one while partial akinesia was seen in 67% of the group. The akinesis resolved by postoperative week one in all cases. This difference in ocular motility between the two groups was statistically significant (p=0.002). There were no adverse events such as infections complications or wound healing complications in either group.

In certain embodiments, patients receiving triamcinolone with bupivacaine and cefazolin had significantly less pain and required less oral analgesics in the first 24 hours following surgery than patients receiving bupivacaine and cefazolin alone. This translates into improved patient satisfaction and less dependence on oral pain medication.

Methods

A controlled trial analyzing the effectiveness of a post-surgery retrobulbar block included adult patients undergoing scleral buckling surgery, 20-gauge vitrectomy, or combined scleral buckle-vitrectomy surgery. Patients undergoing 23 or 25-gauge vitrectomy were included only if the case was combined with a scleral buckle. Patients were excluded if they had a history of previous retinal surgery in the study eye, glaucoma, ocular hypertension, an allergy to local anesthetic, penicillin or cephalosporin, pre-existing chronic pain disorders, uveitis, ocular trauma, or impaired periorbital sensation from herpes simplex, zoster, or a corneal graft. In addition, pediatric patients less than 18 years old, patients unable to verbalize the level of pain control, and glaucoma suspects were excluded. Patients provided written informed consent before enrollment and the study had an independent data and safety monitoring board.

Patients were randomized to two groups: Group A received a 3-mL mixture of 1 mL 0.75% bupivacaine (7.5 mg/1 mL), 1 mL cefazolin (100 mg/mL), and 1 mL triamcinolone (40 mg/mL). Group B received a 3-mL mixture of 1 mL 0.75% bupivacaine (7.5 mg/mL), 1 mL cefazolin (100 mg/mL), and 1 mL of balanced salt solution. A blunt cannula was used to deliver the injections into the retrobulbar space at the conclusion of the surgery after the conjunctiva was closed. The surgeon estimated the volume of reflux and, conversely, assessed the amount of block entering the retrobulbar space was estimated. This was recorded as 0, 25%, 50%, 75%, 100% delivery. Patients were masked to the study medication received. Various intraoperative details were recorded including the duration of surgery, amount and time of preoperative retrobulbar block administered, amount and time of supplemental block given, type of anesthesia, procedures performed, placement of scleral buckle, vitrectomy gauge, and placement of conjunctival sutures. The primary outcome was the pain score on postoperative day 1. The score was reported by the patient, based on a visual analog pain scale, and recorded from 0-10; 10 being the worst. Secondary outcome measures included the total reported oral analgesic intake during the first 24-hours following surgery. Ocular motility was measured on postoperative day 1. Sample size calculations based on the independent group t-test and a significance level of 0.05 with a power of 0.8 indicated that a population size of 29 patients per group would be required to determine a meaningful (1.5 step) difference between groups.

Sixty patients were enrolled and completed this study. Thirty patients were randomly assigned to receive postoperative retrobulbar injections with triamcinolone in Group A and 30 patients to postoperative retrobulbar injections without triamcinolone in Group B. Mean age of the cohort was 52.7 (standard deviation: 17.7, range 18 to 89) years. There were 40 males (67%) and 20 females (33%). There was an equal distribution of scleral buckles, 20-gauge vitrectomy, conjunctival suture placement, and general anesthesia amongst the two groups. There was no statistically significant difference in surgery duration, amount of preoperative block administered, amount of intraoperative supplemental block administered, or postoperative retrobulbar injection reflux between the groups. In addition, there was no difference in average time between administration of postoperative injections and the postoperative day one visit.

On postoperative day one, the mean visual analog pain score was 1.8±2.2 in group A and 3.7±2.8 in group B (p=0.03). On average, patients in group A required 666±363 mg of acetaminophen, 0.6±1.7 mg hydroxycodone, and 5.3±4.0 mg oxycodone in the first 24 hours following surgery. In comparison, patients in group B required 725±571 mg of acetaminophen, 2.9±3.5 of hydroxycodone, and 8.5±6.8 mg of oxycodone. Patients in group A required less pain medication than those in group B during the initial 24 hours following surgery (p=0.04). Full ocular motility was seen in 100% of the group A eyes on postoperative day one while partial akinesia was seen in 67% of the group. The akinesis resolved by postoperative week one in all cases. This difference in ocular motility between the two groups was statistically significant (p=0.002). Mean intraocular pressure measurements in group B eyes were 25±8 on postoperative day one, 13±4 on postoperative week one, and 13±2 on postoperative month one. Mean intraocular pressure measurements in group A were 23±8 on postoperative day one, 14±4 on postoperative week one, and 14±1.4 on postoperative month one. No differences were observed between the groups in intraocular pressure measurements at any of these points in time (p=0.44, 0.96, and 0.49; respectively). There were no adverse events such as infections complications or wound healing complications in either group.

1. A method of preventing eye pain comprising administering a local anesthetic in combination with a steroid to the space around the eye in a subject in need thereof.

2. The method of claim 1, wherein administration is performed after eye surgery.

3. The method of claim 1, wherein the of the space around the eye is selected from periorbital space, retrobulbar block, peribulbar block, orbicularis oculi muscle, and in front of the tragus of the ear.

4. The method of claim 1, wherein the local anesthetic is selected from bupivacaine, xylocaine, proxymetacaine, paracaine, and tetracaine, combinations, salts, or prodrugs thereof.

5. The method of claim 1, wherein the steroid is selected from triamcinolone, triamcinolone diacetate, triamcinolone acetonide, triamcinolone hexacetonide, cortisone, hydrocortisone, prednisone, prednisolone, methylprednisolone, fludrocortisone, beclomethasone, dexamethasone, and betamethasone combinations, salts, or prodrugs thereof.

6. The method of claim 1 further comprising administering an antibiotic as the third component.

7. The method of claim 1 further comprising administering adrenaline and/or hyaluronidase.

8. The method of claim 1, wherein the subject is diagnosed with retinal detachment, proliferative vitreoretinopathy, or retinoschisis.

9. A method of reducing postoperative eye pain comprising administering and effective amount of bupivacaine, salt or prodrug thereof in combination with triamcinolone, salt or prodrug thereof, and an antibiotic into an area located behind the globe of the eye in a subject in need thereof.

10. The method of claim 9, wherein triamcinolone, salt or prodrug thereof is triamcinolone acetonide.

11. The method of claim 9, wherein the subject is diagnosed with retinal detachment, proliferative vitreoretinopathy, or retinoschisis.

12. The method of claim 9, wherein 3 to 9 mg of bupivacaine is administered.

13. The method of claim 9, wherein 90 to 110 mg of cefazolin is administered.

14. The method of claim 9, wherein 20 to 60 mg of triamcinolone is administered.
